(19) United States
(12) Patent Application Publication
(10) Pub. No.: US 2004/0265289 A1
(43) Pub. Date: Dec. 30, 2004

(54) Composition

(76) Inventors: Dieter Elsser, Bargum (DE); Lars Wexoe Petersen, Muskego (DK); Carsten Bjorn Hansen, Knebel (DK); Gerhard Schwarze, Ost-Bargum (DE)

Correspondence Address: Scott A McCollister, Fay Sharpe Fagan Minnich & McKee, 1100 Superior Avenue, 7th Floor, Cleveland, OH, 44114-2518, US

(21) Appl. No.: 10/482,062

(22) PCT Filed: Jun. 26, 2002
(86) PCT No.: PCT/IB02/03259

(30) Foreign Application Priority Data
Jun. 27, 2001 (GB) 0115679.3

(51) Int. Cl. A61L 9/04; A61K 9/14; C12N 1/20

(52) U.S. Cl. 424/93.45; 435/252.9; 424/46

(57) ABSTRACT

A combination is described. The combination comprises: (a) a product for animal use or consumption; and (b) a composition comprising a viable micro-organism and an antimicrobial agent, wherein said antimicrobial agent has been produced by said viable micro-organism.

### FIELD OF INVENTION

The present invention relates to a composition.

In particular, the present invention relates to a composition suitable for preparing or treating products for animal use or consumption that may be contaminated by a microbe. For some embodiments the animal is a human.

### BACKGROUND TO THE INVENTION

Throughout history the prevention of food spoilage and food poisoning has been attempted, often through trial and error. The early attempts have resulted in the adoption of such food preservation methods as the drying, salting and/or smoking of foods in order to preserve them. Only in recent history has food preservation been placed upon a scientific foundation. For example, in the nineteenth century, the work of such scientists as Louis Pasteur and Robert Koch elucidated the bacterial causes of food poisoning and spoilage and provided new methods of identifying pathogenic bacteria and or preserving food.

Several physical, chemical, and biological processes and agents are employed by present food technologists to preserve food and prevent the transmission of disease via foodstuffs. In addition to such processes as irradiation, fermentation, pasteurisation, control of temperature, pH and/or water activity, a plethora of chemical agents exist. These agents include antioxidants to prevent chemical degradation of food, as well as compositions which kill or inhibit deleterious bacteria and/or other microbes thereby preserving food i.e. preventing both spoilage and the transmission of disease. Commonly employed antimicrobial chemical agents include nitrites, nitrates, sulphur dioxide, sulfites, and acids such as acetic, propionic, lactic, benzoic, and sorbic acid and their salts, wood smoke and liquid smoke, and antibodies such as natamycin and nisin.

In the food processing industry the prevention of food poisoning is of paramount importance. In most countries the concern for food safety has led to heavy regulation in the food industry to ensure public health. Also, considerable resources are invested by the manufacturers of processed food to ensure the safety of their products. However, despite these efforts, food poisoning still occurs. Many instances of food poisoning are attributed to bacteria such as Salmonella, Clostridium, and Staphylococcus among others.

Of rising concern is the relatively recent discovery in the food processing industry of widespread Listeria contamination of poultry and processed foods such as wieners, other sausages, cheese, dairy products including novelty ice cream, and seafood. Of particular concern is the recent evidence that pasteurised and fully cooked processed foods are being contaminated with microbes such as Listeria monocytogenes following cooking or pasteurisation and prior to packaging for point of sale. Such contamination is typically surface contamination which is believed to be caused by the contact of microbes with food surfaces subsequent to heat treatment (i.e. cooking or pasteurisation). Microbes such as Listeria may be airborne (i.e. carried by dust) or present on food contact surfaces such as processing equipment.

Several outbreaks of food poisoning have been reported worldwide in which the causative agent is suspected to be or has been identified as Listeria contaminated food. Outbreaks of listeriosis (infection by Listeria bacteria) in humans have been reported in Massachusetts, Calif., and Pennsylvania in the U.S.A and also in Canada, and Switzerland. These outbreaks have been attributed to ingestion of Listeria contaminated food such as coleslaw, cheese made from raw milk, surface ripened soft cheeses and salami. Hundreds of people have been affected with a mortality rate of up to about one third of those affected. Particularly susceptible to the disease (which is contagious) are pregnant woman, foetuses, new-born and infant children as well as adults with compromised immune systems e.g. adults under treatment with immunosuppressive drugs such as corticosteriods. Listeriosis is a serious disease which may cause meningitis, spontaneous abortion, and perinatal septicaemia. Although treatable with early diagnosis, untreated listeriosis exhibits a high mortality rate.

More specifically, Listeria monocytogenes produces severe illness in animals and humans. The characteristics of the disease and this species are described in J.Applied Bact. 63:1-11 (1987)). Listeria monocytogenes grows well at refrigeration temperatures and thus the usual means of inhibiting the growth of Listeria monocytogenes by refrigeration is ineffective. Because of this there are problems in the marketplace, an example of which is the published recall of several brands of Listeria contaminated ice cream bars.

The present invention seeks to provide a useful means to prepare or treat products for animal use or consumption that may be contaminated by a microbe.

### PRESENT INVENTION

In a broad aspect, the present invention relates to the use of a composition comprising a viable micro-organism and an anti-microbial agent for preparing or treating products that may be contaminated by a microbe. The present invention also relates to products that have been coated and/or admixed with said compositions.

In one aspect, the present invention relates to compositions comprising viable Lactobacillus plantarum and pediocin produced therefrom for preparing or treating products that may be contaminated by a microbe. The present invention also relates to products that have been coated and/or admixed with said compositions.

In a first broad aspect, the present invention relates to a combination comprising:

(a) a product for animal use or consumption; and

(b) a spray-dried composition comprising a viable microorganism and an antimicrobial agent, wherein said antimicrobial agent has been produced by said viable micro-organism.

In a second broad aspect the present invention relates to a combination comprising:

(a) a product for animal use or consumption; and

(b) a composition comprising a Lactobacillus plantarum and an antimicrobial agent, wherein said antimicrobial agent has been produced by said viable micro-organism.

### STATEMENT OF INVENTION

Aspects of the invention are presented in the accompanying claims and in the following description.

By way of example, in a first aspect the present invention relates to a combination comprising:

(a) a product for animal use or consumption; and

(b) a composition comprising a viable micro-organism and an antimicrobial agent, wherein said antimicrobial agent has been produced by said viable micro-organism.

In a second aspect the present invention relates to a method for killing, inhibiting or preventing the growth of a microbe comprising contacting a product for animal use or consumption with a viable micro-organism and an antimicrobial agent, wherein said antimicrobial agent has been produced by said viable micro-organism.

In a third aspect the present invention relates to the use of a composition comprising a viable micro-organism and an antimicrobial agent, wherein said antimicrobial agent has been produced by said viable micro-organism, in the treatment of a product for animal use or consumption.

In a fourth aspect the present invention relates to the use of a composition comprising a viable micro-organism and an antimicrobial agent, wherein said antimicrobial agent has been produced by said viable micro-organism, for killing, inhibiting or preventing the growth of a microbe.

In a further aspect the present invention relates to an assay for screening for a suitable composition for use in the present invention, said assay comprising contacting a candidate composition with a product for animal use or consumption and determining the extent of antimicrobial activity to said product; wherein said composition comprises a viable micro-organism and an antimicrobial agent.

Some Preferred Aspects

In a preferred aspect, the composition for use in the present invention is used in a concentrated form.

In a more preferred aspect, the composition for use in the present invention is spray-dried and/or resuspended.

In a preferred aspect the viable micro-organism for use in the present invention is from the genus Lactobacillus.

In a preferred aspect the viable micro-organism for use in the present invention is Lactobacillus plantarum.

In a preferred aspect the antimicrobial agent for use in the present invention is effective against a microbe of the genus Listeria.

In a preferred aspect the antimicrobial agent for use in the present invention is effective against Listeria monocytogenes.

In a preferred aspect the antimicrobial agent for use in the present invention is heat-resistant.

In a preferred aspect the antimicrobial agent for use in the present invention is a bacteriocin.

In a preferred aspect the antimicrobial agent for use in the present invention is pediocin and/or nisin.

It is to be understood that where reference is made in the present specification, including the accompanying claims, to ‘a’ viable micro-organism or ‘an’ anti-microbial agent, such reference is meant to include one or more viable micro-organisms or one or more anti-microbial agents, and mixtures thereof, unless it is specifically stated otherwise in the text.

Some Advantages

Advantages of the present invention are presented in the following commentary.

By way of example, the present invention provides a composition comprising a viable micro-organism which produces effective and/or increased amounts of an anti-microbial agent for the preparation and/or treatment of a product which may be contaminated by a microbe.

In addition, the present invention provides an effective composition comprising viable Lactobacillus plantarum and pediocin for the effective preparation and/or treatment of a foodstuff that may be contaminated by Listeria monocytogenes.

Viable Micro-Organism

Suitable viable micro-organisms for use in the present invention include bacteria, moulds and/or yeasts. In particular the present invention may comprise a composition comprising a viable micro-organism which is a bacteria selected from, but not limited to, various strains of the bacteria Lactococcus, Streptococcus, Pediococcus, Enterococcus, Leuconostoc, Carnobacterium, Propionibacterium, Brifidobacterium and Lactobacillus genuses.

In one aspect, the viable micro-organism for use in the present invention is Lactobacillus plantarum.

Antimicrobial Agents

Suitable antimicrobial agents for use in the present invention may be effective against bacteria, moulds and/or yeasts.

In one aspect, the antimicrobial agents for use in the present invention will be heat resistant, i.e. resistant to destruction or inactivation by heat treatment such as cooking or pasteurisation temperatures and times.

As used herein the term “heat resistant” refers to an antimicrobial agent which is capable of being applied to a product and withstands destruction inactivation or losses due to heat treatment, e.g. by pasteurisation or cooking, such that following heat treatment sufficient agent remains which is effective to kill, inhibit or prevent the growth of microbes on the products to which it is applied. It should be understood that partial losses in the amount of agent or agent effectiveness may occur and that partial inactivation may also occur. However, it is sufficient that the remaining active agent be capable of protecting the product against microbes.

Suitable antibacterial agents include those which are effective to kill, inhibit or prevent the growth of gram positive bacteria. Such agents include, but are not limited to Lactococcus-, Pediococcus-, Streptococcus- or Lactobacillus-derived bacteriocin or bacteriocins derived from lactic acid bacteria.

Bacteriocin

In a preferred aspect, the antimicrobial agent is a bacteriocin.

As used herein the term “bacteriocin” refers to an antimicrobial agent of the colicin type (Peter A. Vandenberg: Lactic acid bacteria, their metabolic products and interference with microbial growth; FEMS Microbiology Reviews 12:221-238 (1993)).

Bacteriocins are characterised by lethal biosynthesis by the producing bacterium, intraspecific activity in related species of bacteria, and adsorption to specific receptors on the sensitive bacteria (Tagg, J. R., A. S. Dajani, and L. W Wannamaker, Bacteriol. Rev. 40:722-756-(1976)). Bacteriocins are usually defined as proteins which produce interspecies antagonistic effects. Bacteriocins have been described as being produced by many bacteria, however the bacterial strains inhibited by the bacteriocin are usually related to the strain which produces the bacteriocin (Gonzalez, C. F., and B. S Kunka, Appl. Environ. Microbiol. 53:2534-2538 (1987)).

Examples of bacteriocins for use in the present invention include, but are not limited to, pediocin, sakacin and enterocin and lantiobiotics such as nisin.

It is well documented that bacteriocin produced by Pediococcus bacteria may be used to inhibit the growth of both pathogenic and spoilage microorganisms such as Listeria monocytogenes. Pediococci are lactic acid bacteria which are used as starter cultures in the manufacture of fermented sausages. In the article by Hoover et al, “A Bacteriocin Produced by Pediococcus Species Associated with a 5.5-Megadalton Plasmid”, Journal of Food Protection, Vol. 51, No. 1 pp 29-31 (January, 1988), a bacteriocin produced by Pediococcus species associated with the 5.5 megadalton plasmid is disclosed as effective to inhibit growth of several strains of Listeria monocytogenes. Bacteriocin from various species of Pediococci are known to be effective to inhibit, kill or prevent growth of Listeria monocytogenes. In particular, bacteriocin from Pediococcus acidilactici are known to be effective against Listeria monocytogenes. In the article by Pucci et al, entitled “Inhibition of Listeria Monocytogenes by Using Bacteriocin PA-1 Produced by Pediococcus acidilactici PAC 1.0”, Applied and Environmental Microbiology, Vol.54 No.10, pp 2349-2353 (October, 1988), Pediococcus acidilactici is disclosed as producing a very effective anti-Listeria agent. The strain of Pediococcus acidilactici discussed in the article was designated PAC 1.0 and produced a bacteriocin designated PA-1 which was very effective against a Listeria monocytogenes at initial inoculum levels of 1.2×10² bacteria per milliliter. Bacteriocins produced from either Pediococcus pentosaceus or Pediococcus acidilactici are referred to herein as pediocin.

However, to date, no one has suggested the use of a composition comprising a viable micro-organism and an antimicrobial agent, wherein said antimicrobial agent has been produced by said viable micro-organism, for use in preparing and/or treating a product for animal use or consumption. By way of example, reference can be made to the following teachings.

WO 99/67287 describes a process for producing a spray-dried bacteriocin lacticin 3147 powder which has anti-microbial activity. Such a process comprises inoculating a medium with a lacticin 3147-producing strain of bacteria; fermenting said medium and adjusting the pH of fermentation, followed by inactivation and evaporation of the bacterial fermentate.

WO 96/21216 describes the isolation of a novel bacteriocin and DNA molecule encoding the bacteriocin, having a molecular weight of 4.4 kDa, and uses thereof.

WO 01/05254 discloses antibacterial compositions comprising at least one gram positive bactericidal compound such as pediocin in combination with hop acids, hop acid derivatives, hop resin and hop resin derivatives, for application to the surface of solid food.

U.S. Pat. No. 5,573,800 describes novel antimicrobial compositions comprising a synergistic combination of a Streptococcus- or Pediococcus-derived bacteriocin and a chelating agent against pathogenic bacteria such as Listeria monocytogenes.

EP 0466 244 discloses a composition having improved antibacterial properties comprising a cell wall lysing substance, an antibacterial compound and an adjuvant selected from organic acids acceptable for use in food products or preparations for cosmetic use or personal hygiene or salts thereof.

EP 384 319 describes synergistic combinations of an isolated bacteriocin and a chelating agent.

EP 326 062 describes a method for inhibiting Listeria monocytogenes using a bacteriocin derived from Pediococcus acidilactici.

Preparing the Composition

Compositions for use in the present invention may be in the form of solid or liquid preparations or combinations thereof. Examples of solid preparations include, but are not limited to tablets, capsules, dusts, granules and powders which may be wettable, spray-dried, freeze-dried, lyophilised. Examples of liquid preparations include, but are not limited to, aqueous, organic or aqueous-organic solutions, suspensions and emulsions.

The compositions of the present invention may be freeze-dried or spray-dried by methods known in the art.

However, lyophilisation (freeze drying) is relatively energy consuming, has a lower material throughput and usually requires the concentration of material prior to drying which may remove certain components of the growth medium such as metabolites produced during fermentation. Nevertheless, in some instances, the use of this technique is acceptable.

Preferably the composition of the present invention is spray-dried.

Typical processes for making particles using a spray drying process involve a solid material that is dissolved in an appropriate solvent (e.g. a culture of a micro-organism in a fermentation medium). Alternatively, the material can be suspended or emulsified in a non-solvent to form a suspension or emulsion. Other components, such as an anti-microbial agent, optionally are added at this stage. The solution then is atomised, for example by either rotary (wheel) or nozzle atomizers, to form a fine mist of droplets. The droplets immediately enter a drying chamber where they contact a drying gas. The solvent is evaporated from the droplets into the drying gas to solidify the droplets, thereby forming dry particles. Evaporation of moisture from the droplets and formation of dry particles proceed under controlled temperature and airflow conditions. The particles are then separated from the drying gas and collected.

The operational conditions for spray drying may be selected according to the drying characteristics of the product and the desired properties of the powder.

Advantageously, the process of spray drying as used herein enables large volumes of material to be dried since the large surface area produced by atomization allows a short residence time in the drying chamber.

A further advantage of the process of spray drying as used herein is that many heat sensitive products can be dried at relatively high inlet temperatures without thermal degradation. For example, during drying, heat and mass transfer occurs in the air and vapour films surrounding the droplets and if the particles do not become “over-dry” and evaporation is still taking place, the temperature of the solid particles will not reach the dryer outlet temperature.

Furthermore, the process of spray drying as used herein is advantageous as it produces a substantially homogenous product from materials that consist of several components, wherein each particle of the product is of substantially the same composition.

The process of spray drying therefore provides several advantages over other drying techniques used in the art.

Preferably the compositions for use in the present invention are in the form of concentrates which comprise a substantially high concentration of a viable micro-organism and/or an antimicrobial agent.

Powders, granules and liquid compositions in the form of concentrates may be diluted with water or resuspended in water or other suitable diluents, for example, an appropriate growth medium or mineral or vegetable oils, to give compositions ready for use.

The compositions of the present invention in the form of concentrates may be prepared according to the methods known in the art.

In one aspect of the present invention the product is contacted by a composition in a concentrated form. Preferably, the product is contacted by a spray-dried and/or resuspended composition.

Preparing the Combination

Combinations of the present invention relate to products that have been contacted with a composition effective against a microbe.

As used herein the term “contacted” refers to the indirect or direct application of the composition of the present invention to the product. Examples of the application methods which may be used in the method of the present invention, include, but are not limited to, coating the product in a material comprising the composition, direct application by mixing the composition with the product, spraying the composition onto the product surface or dipping the product into a preparation of the composition.

Where the product of the invention is a foodstuff, the composition of the present invention is preferably applied to the surface. Alternatively, the composition may be included in the emulsion or raw ingredients of a foodstuff, or to the interior of solid products such as meat products, by injection or tumbling. In a further alternative, the composition may be applied as a marinade, breading, seasoning, rub, glaze, colorant mixture, and the like, the key criteria being that the antimicrobial composition be available to the surface subject to bacterial degradation.

Application of Composition.

The compositions of the present invention may be applied to intersperse, coat and/or impregnate a product with a controlled amount of a viable micro-organism and/or an antimicrobial agent. Mixtures of viable micro-organisms and antimicrobial agents may also be used and may be applied separately, simultaneously or sequentially. Chelating agents, binding agents, emulsifiers and other additives may be similarly applied to the product simultaneously (either in mixture or separately) or applied sequentially.

Intended Use For Combination

The optimum amount of the composition to be used in the combination of the present invention will depend on the product to be treated and/or the method of contacting the product with the composition and/or the intended use for the same. The amount of viable micro-organism and/or an antimicrobial agent used in the compositions should be a sufficient amount to be effective and to remain sufficiently effective to kill, inhibit or prevent the growth of a microbe, preferably Listeria monocytogenes for a sufficient length of time. This length of time for effectiveness should extend at least from the time of contacting to, and including, the time of utilisation of the product.

Advantageously, where the product is a foodstuff, the viable micro-organism and/or an antimicrobial agent would remain effective through the normal “sell-by” or “expiration” date during which the food product is offered for sale by the retailer. Preferably, the effective time would extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment.

The composition of the invention may also be applied wherein the product is a packaged product. In this embodiment, at least part of the packaging material may be contacted with a composition according to the present invention. In the alternative, just the packaging may be contacted with a composition according to the present invention.

Other Components

The combination and/or composition of the present invention may include other components. By way of example, other components may be one or more of: other antimicrobial agents such as chitosan or its derivatives, and/or chelating agents, natural or synthetic seasonings, essential oils, and/or flavours, dyes and/or colorants, vitamins, minerals, nutrients, enzymes, binding agents such as guar gum and xanthum gum, buffers, emulsifiers, lubricants, adjuvants, suspending agents, preservatives, coating agents or solubilising agents and the like.

Where the intended use of the combination is for foodstuffs, or preparations for pharmaceutical or cosmetic use, the other components should be suitable for such use.

Product

Suitable products for use in the present invention include, but are not limited to, a foodstuff, cosmetic product, pharmaceutical product or equipment for handling, processing, packaging or storing a foodstuff, cosmetic product or pharmaceutical product.

Any product which is susceptible to microbial degradation may be used in the present invention. These include but are not limited to fruits and vegetables including derived products, grain and grain-derived products, dairy foods and dairy food-derived products, meat, poultry, seafood, cosmetic and pharmaceutical products. In particular, the present invention may be used in connection with seafood, such as fresh fish or smoked fish for example salmon; dairy products such as red smeared cheese, acid curd cheese, semi-soft and hard cheeses, fresh or processed cheese; meat and meat products such as fermented sausages, emulsion sausages, mince and minced meat products, and ham, and ready-to-use vegetables and salads.

Microbe

The present invention may be used against any microbe capable of contaminating a product for animal use or consumption. Examples of microbes which may be killed, inhibited, or the growth of which may be prevented by the combination of the present invention, include, but are not limited to, bacteria such as Listeria, Staphylococci, Bacilli, Clostridii, Lactobacilli, Entercocci, Micrococci, Corynebacteria, Streptococci and Streptomyces species.

In one aspect of the present invention the microbe to be killed, inhibited or the growth of which is to be prevented, is Listeria monocytogenes.

The present invention will now be described only by way of examples, and with reference to the accompanying figures:

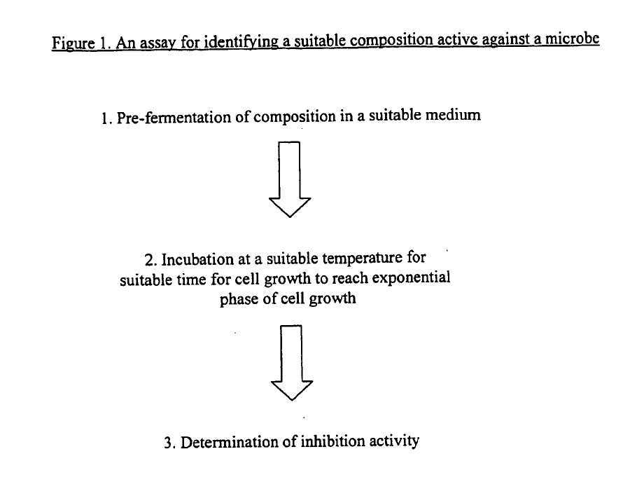

FIG. 1 represents a schematic diagram of an assay for identifying a suitable micro-organism and/or antimicrobial agent against a microbe.

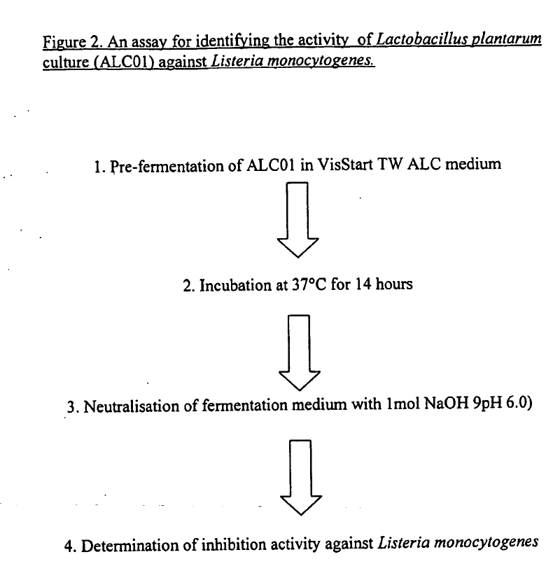

FIG. 2 represents a schematic diagram of an assay for identifying the activity of Lactobacillus plantarum culture (ALC01) against Listeria monocytogenes.

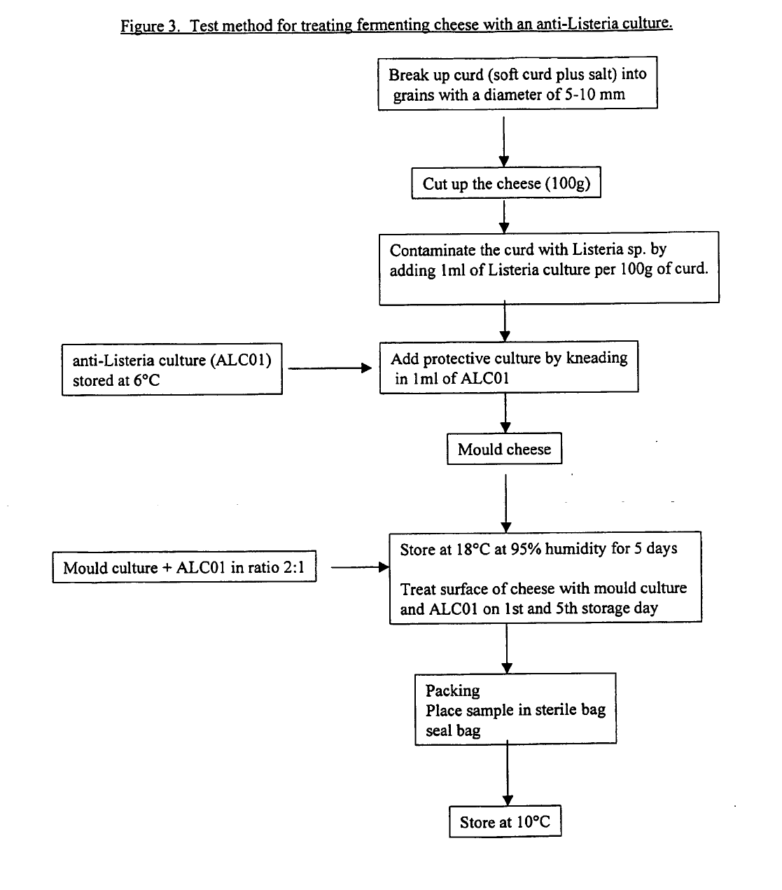

FIG. 3 represents a schematic diagram of a test method for treating acid curd cheese with an anti-Listeria culture.

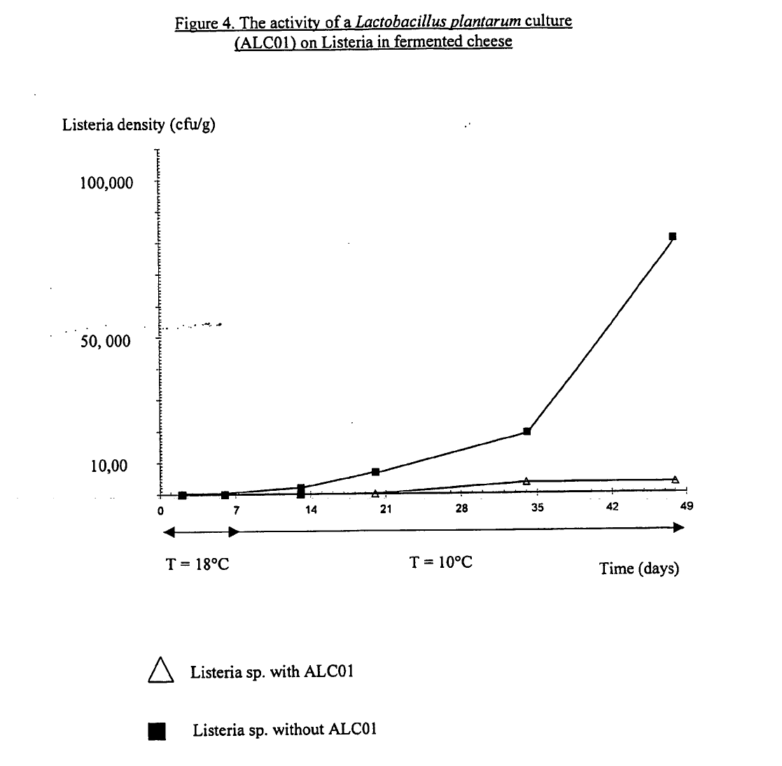

FIG. 4 represents a graph showing the activity of a Lactobacillus plantarum culture (ALC01) on Listeria in acid curd cheese.

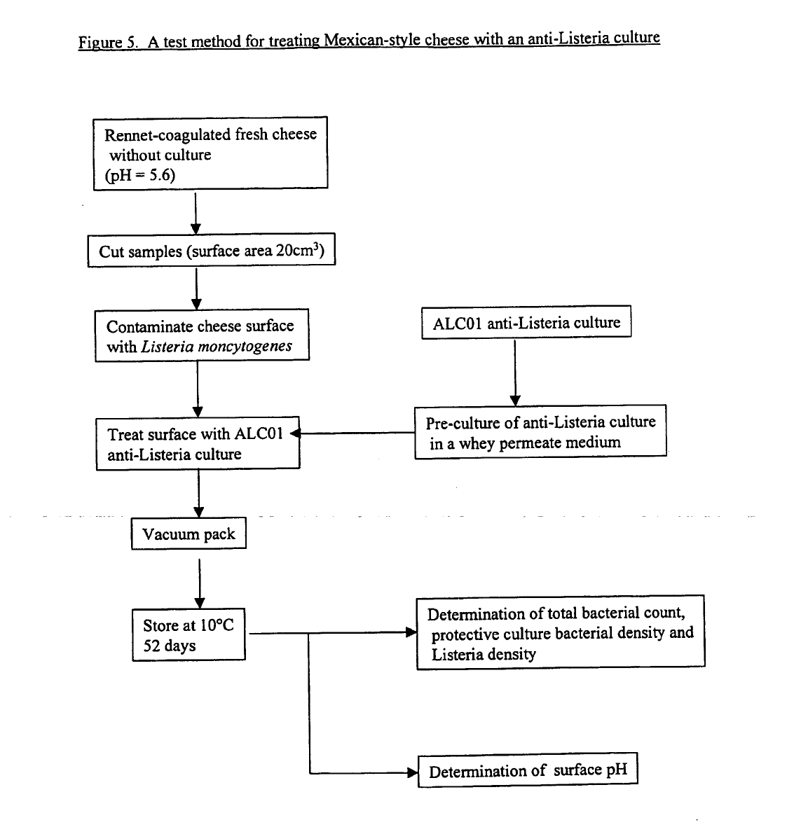

FIG. 5 represents a schematic diagram of a test method for treating Mexican-style cheese with an anti-Listeria culture.

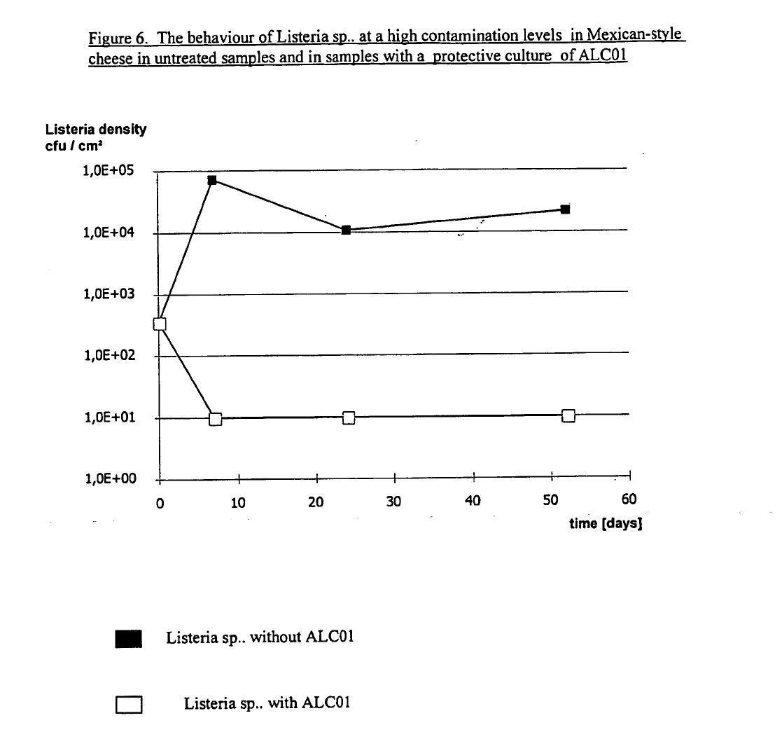

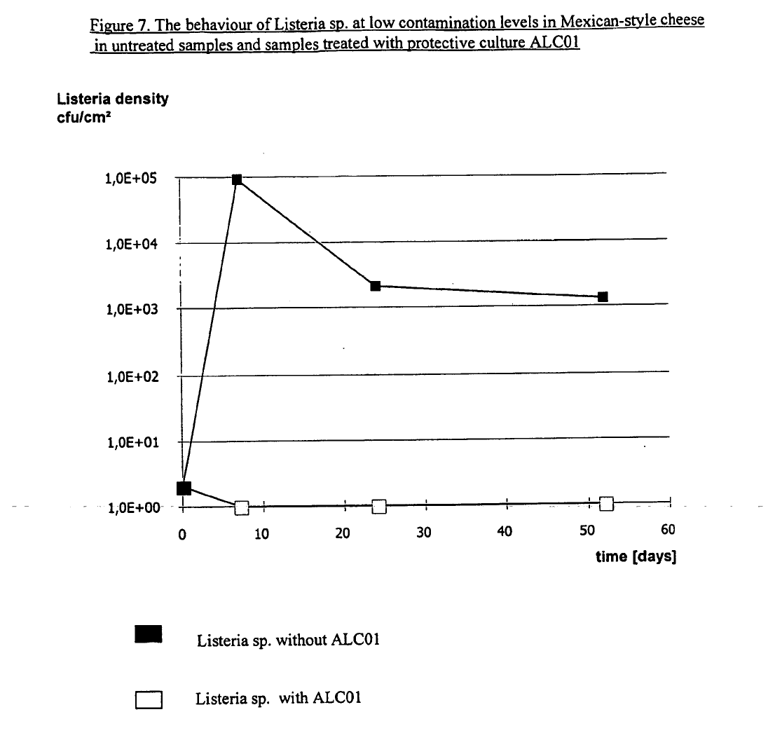

FIGS. 6 and 7 represent graphs showing the activity of a Lactobacillus plantarum culture (ALC01) on Listeria in Mexican-style cheese.

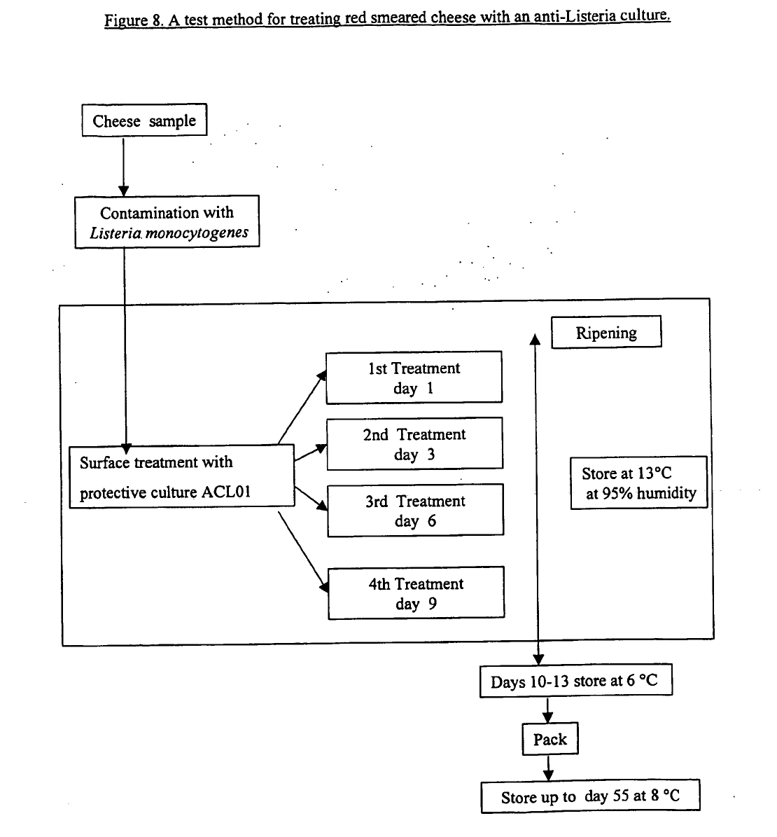

FIG. 8 represents a schematic diagram of a test method for treating red smeared cheese with an anti-Listeria culture.

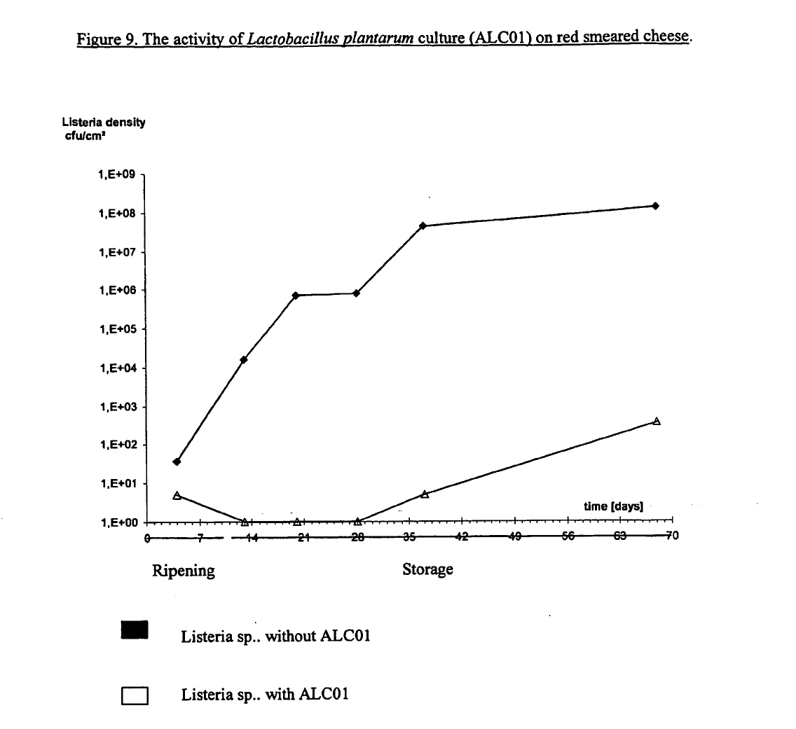

FIG. 9 represents a graph showing the activity of a Lactobacillus plantarum culture (ALC01) on red smeared cheese.

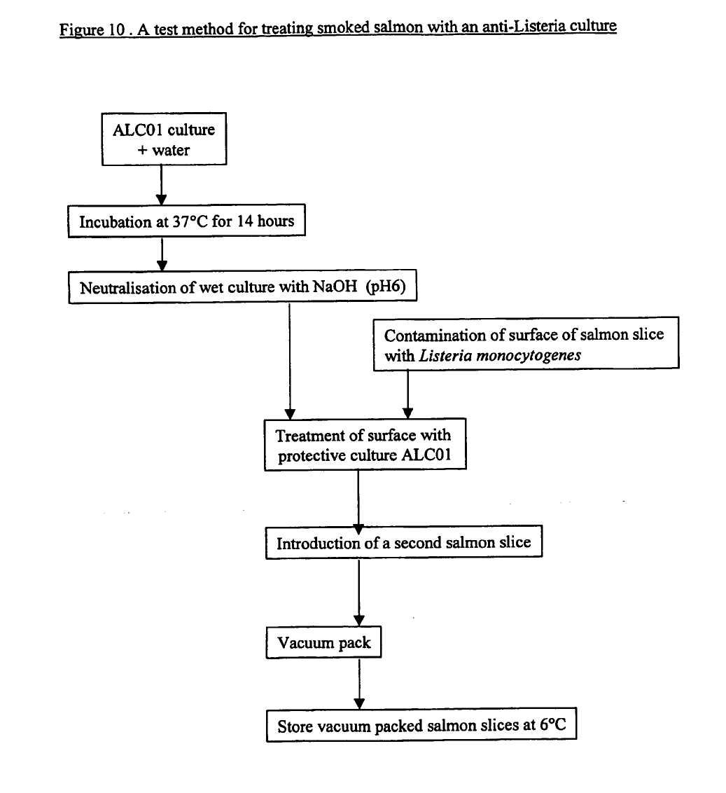

FIG. 10 represents a schematic diagram of a test method for treating smoked salmon with an anti-Listeria culture.

Materials and Methods

1 Microbial Methods

1.1 Preparation of Samples

To determine the Listeria density present in the following test samples, duplicate samples were prepared of portions treated with ALC01 and untreated portions. To this end, a sample portion (25 g) was placed in 225 ml of a diluting agent and homogenised in a Stomacher blender.

Detection of Listeria Cell Counts (1 to 100 cfu/g).

To determine the bacterial density of Listeria, 3×10 ml of a Palcam and/or Fraser medium were inoculated with 1.0 ml and 0.1 ml of the test suspension and incubated at 37° C. for 48 h. If the colour changed (black coloration), it was streaked onto OXFORD-Agar and the colonies were confirmed as Listeria (by microscope, gram-staining or a catalase test).

Detection of Listeria Cell Counts (>100 cfu/g).

Bacterial counts were made with Listeria densities of greater than 100 colony-forming units (cfu) after carrying out the OXFORD-Agar surface method. Typical colonies were determined by gram staining, microscope and a catalase test. The total bacterial count was determined on PC-Agar.

1.2 Preparation of the Composition.

1.2.1 Fermentation Method

A pre-fermenting culture of Lactobacillus plantarum (ALC01) was prepared by inoculating a fermenting medium, for example VisStart TWALC, with Lactobacillus plantarum (ALC01) and stored at 37° C. for 14 hours. Following incubation the inhibiting activity (cell mass separation, neutralisation and sterile filtering) of the pediocin present in the fermenting medium was determined against a Listeria species using a well diffusion assay.

1.2.2 Spray-Drying Method

A pre-fermenting culture of Lactobacillus plantarum (ALC01) was prepared by inoculating a fermenting medium, for example Medium7c-k with Lactobacillus plantarum (ALC01) and stored at 37° C. for 10 hours. Following incubation the fermentate was stored at 6° C. The resulting concentrate was then added to a 7.5% solution of maltodextrin (for example Glucidex IT6) and spray-dried.

2. Assay Method for Identifying a Suitable Composition Against a Microbe.

FIG. 1 illustrates the assay method of the present invention for screening for a suitable composition. In the assay method of the present invention, a fermenting medium is inoculated with a composition and stored at a temperature suitable for cell growth for a period of time suitable for the cell growth to reach its exponential growth phase. Following incubation the inhibiting activity (cell mass separation, neutralisation and sterile filtering) of the composition (viable micro-organism and/or antimicrobial agent) in the fermenting medium is determined against a microbe using a well diffusion assay. The composition is placed in the microtitration plate and its inhibiting activity compared with a known anti-microbial standard.

2.1 The determination of the activity of a culture of Lactobacillus plantarum (ALC01).

By way of example, the activity of a culture of Lactobacillus plantarum (ALC01) was investigated using the assay method described in (2) above.

FIG. 2 illustrates an assay method for identifying the activity of Lactobacillus plantarum (ALC01) against Listeria monocytogenes.

Compositions comprising Lactobacillus plantarum and pediocin were prepared as described in section 1.2 above and placed in a microtitration plate. The inhibiting activities of the compositions against Listeria were compared with a nisin standard.

Results

Compositions comprising Lactobacillus plantarum (ALC01) and pediocin inhibited the proliferation of Listeria.

3. Test Examples

3.1 Determination of the activity of Lactobacillus Dlantarum culture (ALC01) on Listeria in acid curd cheese.

Method

The method for determining the activity of pediocin produced by a culture of Lactobacillus plantarum (ALC01) on Listeria in acid curd cheese is presented in FIG. 3. Fermentates comprising Lactobacillus plantarum (ALC01) culture were produced as described in section 1.2 above and stored at 6° C.

Samples of curd were broken into pieces and contaminated with Listeria (Listeria monocytogenes). To samples of 100 g of broken curd, 1 ml of ALC01 culture was added and 10 g cheese samples were formed using the contaminated curd. The samples were stored at 18° C. for 5 days at 95% humidity and on days 1 and 5 of storage the samples were further treated with ALC01 culture. On day 6 the samples were placed into sterile bags which were sealed and placed into storage at 10° C. The density of Listeria was determined on days 2 and 6 following contamination and days 7, 14, 28 and 42 following packaging and storage at 10° C.

Aspects of the above method can be summarised in the following test protocol:

Acid curd cheese

| | |
|---|---|
| Day 1: | Determination of contamination density |
| Day 2: | Listeria density at 18° C. |
| Day 6: | Listeria density before packing |
| Day 13: | Day 7 of storage at 10° C. |
| Day 20: | Day 14 of storage at 10° C. |
| Day 34: | Day 28 of storage at 10° C. |
| Day 48: | Day 42 of storage at 10° C. |

The Listeria density was determined according to the protocol described in the Microbial Methods section above.

The surface pH was determined several times for 2 portions of the cheese samples (25 g each) using a surface pH electrode.

Results

The following table 1 illustrates the activity of ALC01 culture on Listeria densities in acid curd cheese.

TABLE 1

| | Test without ALC01 | | Test with ALC01 | |
|---|---|---|---|---|
| | Average Listeria | | Average Listeria | |
| | density | | density | |
| Acid curd cheese | [cfu/g] | pH | [cfu/g] | pH |
| Day 1: Determination of | — | 5.0 | — | 5.0 |
| contamination density | | | | |
| Day 2: Listeria density | 0 | 5.2 | 0 | 5.1 |
| on day 2/18° C. | | | | |
| Day 6: Listeria density | 10 | 7.9 | 0 | 7.6 |
| prior to packaging | | | | |
| Day 13: 7^{th} day of | 875 | 7.9 | 0 | 7.4 |
| storage at 10° C. | | | | |
| Day 20: 14^{th} day of | 6625 | 7.6 | 0 | 7.1 |
| storage | | | | |
| Day 34: 28^{th} day of | 19450 | 7.8 | 1650 | 7.7 |
| storage | | | | |
| Day 48: 42^{nd} day of | 83250 | 8.1 | 1675 | 7.9 |
| storage | | | | |

The results are presented graphically in FIG. 4.

For the tests without ALC01 treatment, even on the packaging day, small Listeria densities of 10 cfu/g were observed. On storage at 10° C., the Listeria density increased substantially; at the end of the storage period, in one sample there were already 1.1×10⁵ Listeria.

Listeria cell counts in samples treated with ALC01 were detected from day 28 (but with lower cell counts).

3.2 Determination of the Activity of Lactobacillus plantarum culture (ALC01) on Listeria in Mexican-Style Cheese.

Method

The method for determining the activity of pediocin produced by a culture of Lactobacillus plantarum (ALC01) on Listeria in Mexican-style cheese is presented in FIG. 5.

A sample of Mexican-style cheese (e.g. rennet-coagulated fresh cheese without culture) was cut into pieces and contaminated with Listeria (Listeria monocytogenes). To this sample, 1 ml of ALC01 culture (as prepared in section 1.2 above) was applied to the surface and the contaminated cheese was vacuum-packed and placed into storage at 10° C. for 52 days.

The Listeria density was determined according to the protocol described in the Microbial Methods section above.

The surface pH was determined several times for 2 portions of the cheese samples (25 g each) using a surface pH electrode.

Results

The results are presented graphically in FIGS. 6 and 7.

Cultures of ALC01 inhibit the proliferation of 0.1 ml Listeria on Mexican-style cheese having a high Listeria contamination level for up to 24 days following contamination.

Cultures of ALC01 inhibit the proliferation of 0.1 ml and 1 ml Listeria on Mexican-style cheese having a low Listeria contamination level for up to 52 days following contamination.

3.3 Determination of the Activity of Lactobacillus plantarum Culture (ALC01) on Red Smeared Cheese Contaminated With Listeria.

Method

The method for determining the activity of pediocin produced by a culture of Lactobacillus plantarum (ALC01) on Listeria in red smeared cheese is presented in FIG. 8.

Cultures of Lactobacillus plantarum (ALC01) were prepared according to the method described in section 1.2 above. The culture was then applied on days 1, 3, 6 and 9 to the surface of a sample of red smeared cheese following contamination with Listeria (Listeria monocytogenes) on day 1. Between applications, the red smeared cheese sample was stored at 13° C. and at a relative humidity of greater than 95%. On day 10 the red smeared cheese sample was placed into storage at 6° C. for three days after which it was packed and put into storage at 8° C. for up to 55 days.

Results

The results are presented graphically in FIG. 9.

3.4 Determination of the Activity of Lactobacillus plantarum Culture (ALC01) on Listeria in Smoked Salmon.

Method

The method for determining the activity of pediocin produced by a culture of Lactobacillus plantarum (ALC01) on Listeria in smoked salmon is presented in FIG. 10.

A slice of smoked salmon was cut and contaminated with Listeria (Listeria monocytogenes) at 50 units per salmon surface. A protective culture of ALC01 culture (as prepared in section 1.2.2 above) was added to water and applied to the surface of the contaminated fish sample. A second slice of smoked salmon was placed on the surface of the contaminated slice and the two slices were vacuum-packed together and stored at 6° C.

Results

The following table 2 illustrates the activity of the protective bacterial ALC01 culture on Listeria in smoked salmon.

TABLE 2
The activity of Lactobacillus Diantarum culture (ALC01) on Listeria
contamination in smoked salmon

| | No ALC01 | | With ALC01 | |
|---|---|---|---|---|
| Test day | 10⁻¹* | 10⁻²** | 10⁻¹ | 10⁻² |
| 0 | 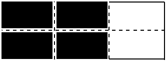 | 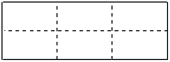 | 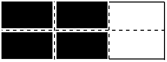 | 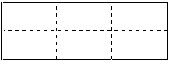 |
| 5 | 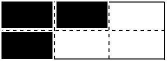 | 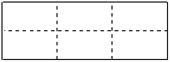 | 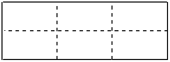 | 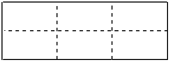 |
| 12 | 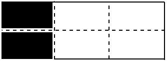 | 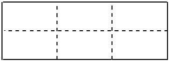 | 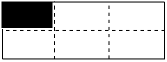 | 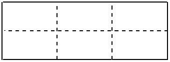 |
| 19 | 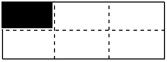 | 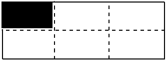 | 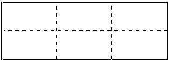 | 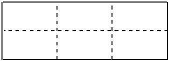 |
| 29 | 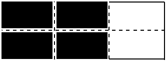 | 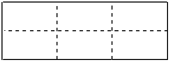 | 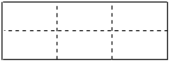 | 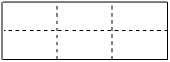 |

*Dilution 10⁻¹1: Homogenisation of whole sample in 1:10 ratio
Enrichment in 1 ml of homogenate
**Dilution 10⁻²: Homogenisation of whole sample in 1:10 ratio
Enrichment in 0.1 ml of homogenate 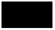
Listeria positive enrichment
Listeria negative enrichment

Only one sample showed any contamination of Listeria, after 12 days storage, in the presence of ALC01. In comparison, samples without the protective culture ALC01 showed contamination up to 19 and 29 days after storage.

Discussion

The tests carried out with an acid curd cheese, Mexican-style cheese, red smeared cheese and smoked salmon clearly show that protective culture ALC01 (as applied pre-fermentation in VisStart TW ALC or spray-dried in Medium7c-k) can substantially minimise the microbe health risk caused by Listeria monocytogenes.

Summary Paragraphs

In summary, aspects of the present invention will now be described by way of numbered paragraphs.

1. A combination comprising:

(a) a product for animal use or consumption; and

(b) a composition comprising a viable micro-organism and an antimicrobial agent, wherein said antimicrobial agent has been produced by said viable micro-organism.

2. A combination according to paragraph 1 wherein said viable micro-organism is from the genus Lactobacillus.

3. A combination according to paragraph 1 or 2 wherein said viable micro-organism

5 is Lactobacillus plantarum.

4. A combination according to any preceding paragraph wherein said antimicrobial agent is effective against a microbe of the genus Listeria.

5. A combination according to any preceding paragraph wherein said antimicrobial agent is effective against Listeria monocytogenes.

6. A combination according to any preceding paragraph wherein said antimicrobial agent is heat-resistant.

7. A combination according to any preceding paragraph wherein said antimicrobial agent is a bacteriocin.

8. A combination according to any preceding paragraph wherein said antimicrobial agent is pediocin and/or nisin.

9. A combination according to any preceding paragraph wherein said composition is in a concentrated form.

10. A combination according to any preceding paragraph wherein said composition is spray-dried and/or resuspended.

11. A method for killing, inhibiting or preventing the growth of a microbe comprising contacting a product for animal use or consumption with a composition as defined in any one of the preceding paragraphs.

12. A method according to paragraph 11 wherein said viable micro-organism is Lactobacillus plantarum.

13. A method according to paragraph 11 or 12 wherein said antimicrobial agent is effective against Listeria monocytogenes.

14. A method according to paragraph 11, 12 or 13 wherein said antimicrobial agent is pediocin and/or nisin.

15. A method according to paragraphs 11 to 14 wherein said composition is in a concentrated form.

16. A method according to any one of paragraphs 11 to 15 wherein said composition is spray-dried and/or resuspended.

17. The use of a composition comprising a viable micro-organism and an antimicrobial agent, wherein said antimicrobial agent has been produced by said viable micro-organism, in the treatment of a product for animal use or consumption.

18. The use of a composition comprising a viable micro-organism and an antimicrobial agent, wherein said antimicrobial agent has been produced by said viable micro-organism, for killing, inhibiting or preventing the growth of a microbe.

19. An assay for screening for a suitable composition, said assay comprising contacting a candidate composition with a product for animal use or consumption and determining the extent of antimicrobial activity to said product; wherein said composition comprises a viable micro-organism and an antimicrobial agent.

All publications mentioned in the above specification are herein incorporated by reference. Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in microbiology or related fields are intended to be within the scope of the following claims.

1. A combination comprising:
(a) a product for animal use or consumption; and
(b) a spray-dried composition comprising a viable micro-organism and an antimicrobial agent, wherein said antimicrobial agent has been produced by said viable micro-organism.

2. A combination according to claim 1 wherein said viable micro-organism is from the genus Lactobacillus.

3. A combination according to claim 1 wherein said viable micro-organism is Lactobacillus plantarum.

4. A combination according to claim 1 wherein said antimicrobial agent is effective against a microbe of the genus Listeria.

5. A combination according to claim 1 wherein said antimicrobial agent is effective against Listeria monocytogenes.

6. A combination according to 1 claim wherein said antimicrobial agent is heat-resistant.

7. A combination according to claim 1 wherein said antimicrobial agent is a bacteriocin.

8. A combination according to claim 1 wherein said antimicrobial agent is pediocin and/or nisin.

9. A combination according to claim 1 wherein said spray-dried composition is in a concentrated form.

10. A combination according to 1 claim wherein said spray-dried composition is resuspended.

11. A method for killing, inhibiting or preventing the growth of a microbe comprising contacting a product for animal use or consumption with a spray-dried composition as defined in claim 1.

12. A method according to claim 11 wherein said viable micro-organism is Lactobacillus plantarum.

13. A method according to claim 11 wherein said antimicrobial agent is effective against Listeria monocytogenes.

14. A method according to claim 11 wherein said antimicrobial agent is pediocin and/or nisin.

15. A method according to claim 11 wherein said spray-dried composition is in a concentrated form.

16. A method according to claim 11 wherein said spray-dried composition is resuspended.

17. The use of a spray-dried composition comprising a viable micro-organism and an antimicrobial agent, wherein said antimicrobial agent has been produced by said viable micro-organism, for killing, inhibiting or preventing the growth of a microbe.

18. The use of a spray-dried composition comprising a viable micro-organism and an antimicrobial agent, wherein said antimicrobial agent has been produced by said viable micro-organism, for killing, inhibiting or preventing the growth of a microbe.

19. An assay for screening a suitable spray-dried composition, said assay comprising contacting a candidate spray-dried composition with a product for animal use or consumption and determining the extent of antimicrobial activity to said product; wherein said spray-dried composition comprises a viable micro-organism and an antimicrobial agent.

20. A combination comprising:
(a) a product for animal use or consumption; and
(b) a composition comprising a Lactobacillus plantarum and an antimicrobial agent, wherein said antimicrobial agent has been produced by said viable micro-organism.

21. A combination according to claim 20 wherein said composition is spray-dried.

22. A combination according to claim 20 wherein said antimicrobial agent is effective against a microbe of the genus Listeria.

23. A combination according to claim 20 wherein said antimicrobial agent is effective against Listeria monocytogenes.

24. A combination according to claim 20 wherein said antimicrobial agent is heat-resistant.

25. A combination according to claim 20 wherein said antimicrobial agent is a bacteriocin.

26. A combination according to claim 20 wherein said antimicrobial agent is pediocin and/or nisin.

27. A combination according to claim 20 wherein said antimicrobial agent is in a concentrated form.

28. A combination according to claim 20 wherein said antimicrobial agent is resuspended.

29. Cancel.
